(19) United States
(12) Patent Application Publication
(10) Pub. No.: US 2012/0202223 A1
(43) Pub. Date: Aug. 9, 2012

(54) Ki-67 ASSAY FOR PATIENT IMMUNE SYSTEM STATUS

(76) Inventors: Denis V. Baev, Alghero (IT); Franco Lori, Bethesda, MD (US)

(21) Appl. No.: 13/020,285

(22) Filed: Feb. 3, 2011

(51) Int. Cl. G01N 33/53; C12Q 1/02

(52) U.S. Cl. 435/7.24; 435/29

(57) ABSTRACT

Novel diagnostic assay based on the detection of Ki-67 expression to anticipate the response to anti-HIV therapy

### FIELD OF THE INVENTION

This invention relates to a novel assay for immune system status. The expression of Ki-67 as a proliferation marker in an infected patient's T-cells can be analyzed and the results used to predict the patient's likely disease progression and responsiveness to therapies, including drug treatments and vaccines. This method can be utilized as a guide for an individual patient's therapy management (personalized therapy) over both the short and mid term.

The invention relates to a novel test that quantifies the percentage of proliferated CD3 T cells as an immune monitoring method, and to evaluate the therapy outcome in patients suffering of chronic infections, neoplastic and allergic diseases where the expression level of Ki-67 in T-cells correlates with disease progression.

The proof of principle of the utility of this marker is demonstrated in a representative chronic disease treatment scenario: where individuals who are chronically infected with HIV undergo therapy with antiviral drugs or vaccines. The number of Ki-67+ T-cells before therapy was begun correlated with the patients' reduction of an HIV viral load after therapy commenced better than the currently accepted CD4 counts and CD38 expression levels in two independent cohorts. We found that a level of the baseline Ki-67 expression in CD3, CD4 and CD8 T cell subsets but not CD4 counts or baseline values of CD38 and other activation markers (i.e. CD69, HLA-DR and PD-1), significantly correlated with changes in plasma HIV viral loads after treatment had begun.

A known flow cytometry method can be used to detect Ki-67 expression in whole CD3 cells in donor blood. In the alternative, the Ki-67 level can be detected by ELISA or bead-array methods in lysed CD3 cells sorted from donor blood samples.

This test can be used to monitor the ability of prophylactic and therapeutic approaches to produce changes in the immune system status, or competence, of individuals with HIV/AIDS, and to predict short- and long-term success or failure of treatments, including antiretroviral therapy and immune therapies based on immune modulation and targeted immune amplification, and finally to guide a HIV patient's disease management.

### BACKGROUND OF THE INVENTION

One of the great frustrations in efforts to deal with chronic infections generally in humans, and in the field of treatment of HIV/AIDS in particular is that, while drugs and treatments such as vaccines have been developed that can perform according to the design goals, the design goals, when reached, have not cured the target conditions. As a result, powerful drugs that could both reduce viral replication to undetectable levels ion the blood and increase the number of CD4 cells to a more normal range, all in a relatively short period of time, could not end the disease condition. While these two factors yield some information about a patient's current immune system status, they are insufficient to support the next phase of therapy research. Other disease markers must be developed for further progress to be made. The present inventors have focused on a means to elucidate better, more predictive information about a patient's immune system, by determining a patients' present state of immune system activation. Various markers for immune system activation, namely CD38, CD69, HLA-DR, CD4 counts and changes in plasma HIV viral loads were compared, and baseline Ki-67 expression in CD3, CD4 and CD8 T cell subsets was found to be more predictive than CD4 counts or baseline values of CD38 and other activation markers (i.e. CD69, HLA-DR).

An individual patient's degree of immune system activation plays a major role in determining the level of HIV replication and in the eventual depletion of CD4 cells that results in progression of the disease. In 1981 the very first observation of the increased expression of a protein that functions as a T cell activation maker, T10 (designated later on as CD38) in Pneumocystis carinii pneumonia patients was published (Masur H et al., The New England Journal of Medicine, 1981). CD38 is a glycoprotein found on the surface of many immune system cells including CD4+, CD8+, B and natural killer cells. Masur et al. connected CD38 with the appearance of multiple cases of opportunistic infections that occur in acquired immune deficiency patients. The critical role of immune activation in HIV disease progression and in survival of HIV-infected patients was reported first in 1999 (Janis V. Giorgi et al., The Journal of Infectious Diseases 1999;179:859-70). Giorgi and colleagues reported that high levels of expression by T Cells of both CD38 and HLA-DR (cell surface receptor proteins involved in the binding of antigents) are associated with shorter survival of HIV-infected patients and could be considered as possible predictive markers for HIV disease progression. These are said to be more likely predictors than either than plasma HIV concentrations or tests for the virus usage of CCR5 and CXCR4 chemokine receptors. Multiple current observations have reported that CD38 expression could be considered as a promising marker to evaluate progression of the disease with recommendations to use this marker as a diagnostic value (Coetzee L M, Cytometry B Clin Cytom. 2009 November;76(6):375-84.) in routine clinical testing.

The present inventors have explored the use of another protein. The Ki-67 protein (also known as MKI67) is a cellular marker strictly associated with cell proliferation. During interphase (collectively, phases G₁, S and G₂), Ki-67 can be exclusively detected within the cell nucleus, and during mitosis most of the protein is located on the surface of the chromosomes. Ki-67 protein is present during all active phases of the cell cycle (G1, S, G2, and mitosis), but is undetectable in resting cells (G0). Maximal virus replication occurs in G1 and S phases (Foli A. et al., J Infect Dis. 2007 Nov. 1;196(9):1409-15.), when Ki-67 is also up-regulated, so that the only phase when Ki-67 is present and maximal virus replication is not occurring is the relatively short, intermediate, G₂ phase.

The persistent immune activation characteristic of chronic infections, and very particularly evident in HIV infection eventually results in the depletion of CD4 T cells because it progressively erodes the naive T cell pool. Compelling evidence of the role of Ki-67 in HIV-disease progression was reported by M. Lederman and colleagues (Sieg S F, Lederman M M et al., J Infect Dis. 2005 Jul. 1;192(1):62-70.)

Thus, the inventors posited that the comparison of activation markers might yield better ways to evaluate the clinical status of HIV-infected patients and to monitor the effects of anti-HIV therapy.

### BRIEF DESCRIPTION OF THE DRAWINGS

FIG. 1 Cohort 1: VS411 study. Ki-67 expression is dramatically up-regulated in HIV infected patients (1B) up to 2 . . . 10% of total T cells compared to 0.05 . . . . 0.5% in non-infected individuals (1A) and decreases to 0.5 . . . 2% after 24 weeks of VS411 AV-HALT treatment (1C). The Ki-67 decrease is statistically significance (p<0.001) by paired Student's t-test. Patient's PBMCs were stained for surface T cell markers and for Ki-67 by intracellular staining and analyzed on BD LSR-II flow cytometer. One representative dot-plot out of 10 subjects in FIG. 1A and out of 32 subjects in FIGS. 1B and 1C is presented, respectively.

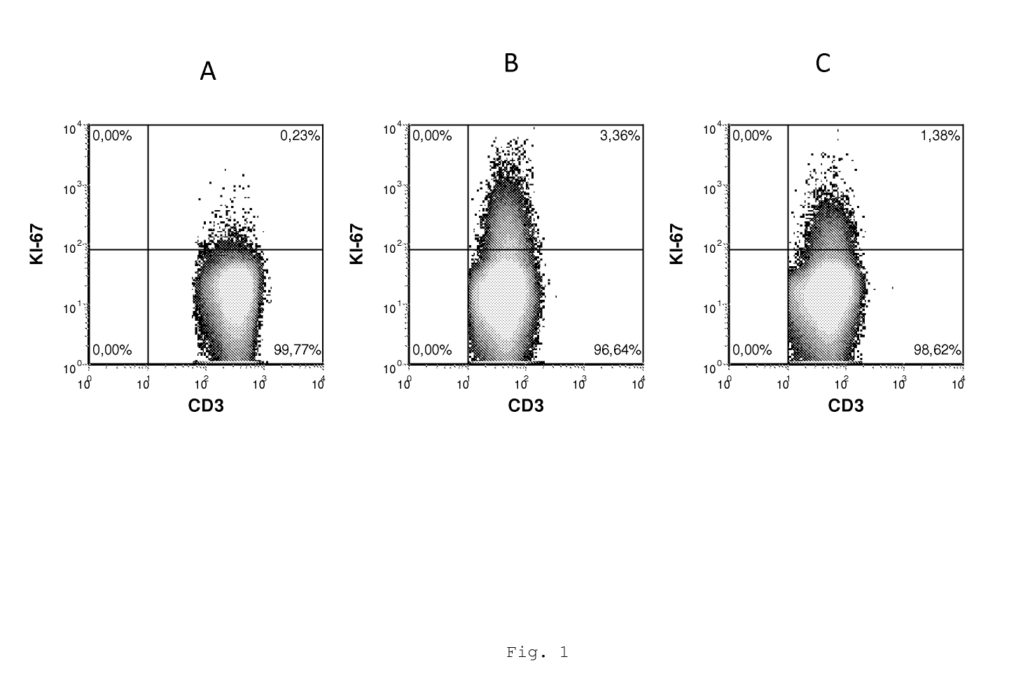

FIG. 2 Cohort 1: VS411 study. The expression of Ki-67 is strongly correlates with the reduction of HIV viral load. 28 subjects from Phase 2A VS-411 study were analyzed for Ki-67 expression on CD3+, CD3+CD4+ and CD3+CD8+ T cells and the Pearson's bi-variate correlation was applied between the baseline values of Ki-67 positive cells percentage and HIV viral load decrease from baseline to last day of anti-HIV treatment. The correlations were characterized by r²=0.272 for the CD3+ subset (p=0.004)(2A), r²=0.19 for CD3+CD4+ (p=0.018)(2B) and r²=0.21 for CD3+CD8+ (p=0.013)(2C) subsets, respectively.

FIG. 3 Cohort 2: RIGHT702 study. Ki-67 baseline expression in CD3 cells strongly correlates with HIV viral load decrease for short-term and long-term therapy outcomes. T cells from 7 patients from RIGHT702 study were analyzed for Ki-67 by flow cytometry analysis and correlation was found with baseline values of Ki-67 and HIV viral load changes on week 12 (A) and week 24 (B) with r²=0.852 (p=0.003) and r²=0.945 (p=0.001), respectively

### SUMMARY OF THE INVENTION

We describe here a diagnostic assay for Ki-67 monitoring, the steps comprising:
a. Isolation of peripheral blood mononuclear cells (PBMC)
b. Staining of T-cells among the PBMC for surface markers
c. Intracellular staining of T cells for Ki-67
d. Measurement of the percentage of Ki-67 positive cells by flow cytometry. Does this yield actively dividing immune system cells v. quiescent immune system cells?

The diagnostic assay proof of principle was demonstrated in a chronic disease, namely HIV-1/AIDS. In this assay, the cells may be frozen PBMCs derived from HIV-infected patients and Ki-67 expression in different T cell subsets is detected by the intracellural staining with anti-Ki-67 antibodies and analyzed by multi-color flow cytometry, as demonstrated in our examples for two independent cohorts of VS411 Phase2A and RIGHT702 studies, or freshly isolated PBMCs, or sorted CD3 cells. Sorted cells could by lyzed and the Ki-67 expression could also be detected by ELISA or any other method. In the present application, Ki-67 was analyzed in CD3, CD4 and CD8 T cells. For HIV-1, a dramatic increase in number of circulating Ki-67 positive T cells is a particular indicator for disease progression.

This assay relies on detection of Ki-67 in patients PBMC before the anti-HIV treatment is started.

To develop the assay we have started with a chronic infection that represents a world pandemic emergency: HIV infection. An advantage of this assay is that it removes a major barrier to drug development for chronic infections. In this disease, the development of effective therapy has been hampered for the past 25 years due to the absence of a rapid test that can correlate immune system markers with disease progression in a case where the anti-HIV therapy is applied. Here we describe a test that yields a quantitative determination of a specific marker in T cells that can predict the therapy outcome in HIV infection.

Another advantage of the invention is that it modifies the standard flow cytometry assays, such as CD4 enumeration and analysis of CD38 expression, which did not yield a good predictive potential (CD4counts) for the therapy outcome and good correlation (CD38) with HIV-1 viral load changes

To investigate the differences between the newly developed Ki-67 assay and the standard approaches we tested the PBMC from 32 HIV-1-infected individuals from Phase2A VS411 study with both assays. Surprisingly, we found that a level of the baseline Ki-67 expression in CD3, CD4 and CD8 T cell subsets but not CD4 counts or baseline values of CD38 and other activation markers (i.e. CD69, HLA-DR), significantly correlated with changes of plasma HIV viral loads. The analysis of 7 additional PBMC samples from an independent cohort of HIV-1-infected individuals from RIGHT702 study confirmed the correlation between the baseline Ki-67 expression and decrease of HIV-1 viral load in all patients. These results demonstrate that the quantity of Ki-67+ T-cells detected with this test can be used as predictive tool to determine the immunological status of chronically-infected patients and to predict the therapy outcome. In addition, a certain range of baseline number of Ki-67+CD3+ cells can be correlated with a certain range of decrease of viral load (“VL”). For example, the percentage of CD3+Ki-67+ cells below 2-2.5% were associated with VL decrease for more than 1.5 logs.

The Ki-67-based assay provides an immune monitoring tool for patients suffering from HIV/AIDS to (1) determine their specific immunological status; (2) to predict their individual disease progression; (3) for individual patient management (for example for determining when to start therapy or how intensely to monitor a patient); and (4) for the design, testing and screening of therapies based on anti-viral drugs, immune modulation, targeted immune amplification, and prophylactic and/or therapeutic vaccines.

### DESCRIPTION OF INVENTION

Although several lines of evidence suggests that circulating Ki-67+T-cells play a critical role as a reservoir of HIV infection in humans and SIV in the macaque model, the precise immune correlates of HIV progression and responsiveness to the therapy remain to be identified.

The flow cytometry is currently widely used to identify HIV-specific effects on T cells in humans. Current approaches include CD4 enumeration and monitoring of activation markers, like CD38 and HLA-DR, which have a predictive potential in terms of disease progression but not in the evaluation of possible anti-HIV therapy outcome. Conversely, the present invention quantifies the Ki-67 positive T cells more likely representing the HIV-related pool of T cells.

In this assay peripheral blood mononuclear cells are first collected from donors or derived from criopreserved blood samples and stained for surface T cell markers and for Ki-67 followed by the flow cytometry analysis of the Ki-67 expression level.

In the present application, the percentage of Ki-67+ T cells quantified by flow cytometry was compared with other activation markers such as CD38, CD69, PD-1 and HLA-DR to discern which parameter correlates with the viral load decrease in chronically HIV-infected patients receiving the anti-HIV therapy.

We analyzed the relationships among various baseline expression levels of activation markers and plasma viral loads and viral load decreases and found that the baseline percentage of Ki-67+ in CD3, CD4 and CD8 subsets but not other markers have a strong and statistically significant correlation with HIV viral load decrease.

The present study is the first to demonstrate that the baseline percentages of Ki-67+ cells in CD3, CD4 and CD8 subsets inversely correlated with the decrease of plasma HIV viral load in HIV-infected individuals undergo the AV-HALT anti-viral therapy.

These results are consistent with the observation that there is a direct connection between the number of circulating S-phase (Ki-67+) T cells and the level of plasma HIV viremia as it was reported by many investigators. Results from a recent study also provides the evidence that even the baseline expression of Ki-67 in total CD3 subset could serve as a predictive marker for the changes of HIV plasma levels in patients receiving anti-viral therapy.

In the RIGHT702 study the baseline percentage of Ki-67+ T cell showed even better potential to predict both short-term (12 weeks) and long-term (24-48 week) therapy outcomes in HIV-infected patients.

In conclusion, our finding represents the first evaluation of predictive potential of Ki-67 marker in HIV-infected individuals for the therapy outcome evaluation. The lower percentage of Ki-67+ T-cells is associated with higher decrease in plasma viremia and better therapy outcome.

The Ki-67 assay will be useful to evaluate the status of the immune system and the recovery of its function in patients treated with antiretroviral drugs as well as to assess the efficacy of prophylactic and therapeutic vaccines.

### EXAMPLES

The following examples illustrate the practice of various aspects of the present inventions. They do not limit the inventions, or the claims, which follow them

### Example 1

### Cohort 1, VS411 Study

Methods: Blood samples from 32 patients receiving VS411 (a novel combination of an antiviral and a cytostatic drug) during a Phase 2A, dose finding study were analyzed by 10-color flow cytometry for activation and proliferation T cell markers. Pearson correlation analysis was performed for baseline and on-treatment values of T cell markers expression versus HIV viral load (VL) and VL changes.

Results: After treatment had begun, KI67 expression in CD3+ cells level was reduced from 3.1±1.68% to 2.18±0.9% (p=0.0012). PD-1, CD38 and HLA-DR expressions were down-regulated from 15.33±6.12% to 11.32±5.01%, from 58.48±14.81% to 52.8±14.25%, and from 27.5±10.01% to 22.2±7.55%, respectively, with p values <0.005. In CD4+ T cell subset the Ki-67 expression level was reduced from 3.23±1.89% to 2.42±1.11% (p=0.016). PD-1 and HLA-DR expressions were down-regulated from 15.3±7.53% to 11.67±6.59% and from 11.94±5.54% to 9.56±4.45%, respectively, with p values <0.01. In CD8+ T cell subset the Ki-67 expression level was reduced from 2.79±1.85% to 1.89±1.09% (p=0.0025). PD-1, CD38 and HLA-DR expressions were down-regulated from 16.75±8.11% to 11.93±6.57%, from 59.13±15.5% to 50.1±15.01% and from 36.03±12.69% to 30.02±10.68, respectively, with p values <0.005.

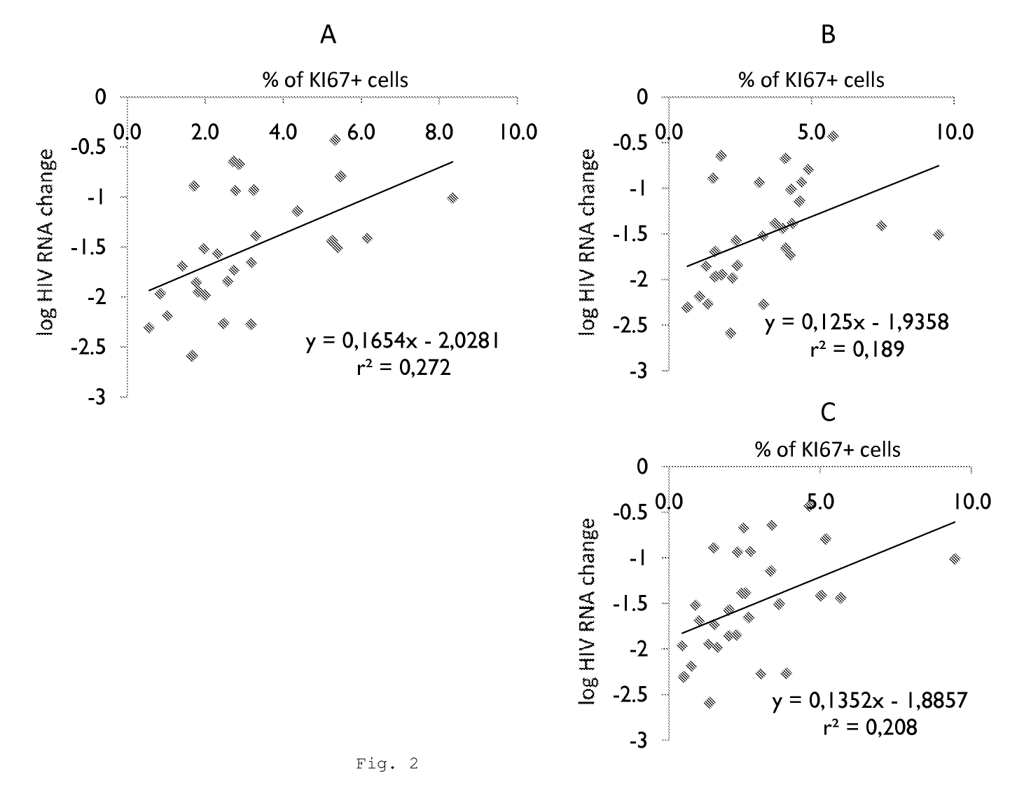

Most importantly, a negative correlation between baseline expression levels of Ki-67 in either CD3, CD4 or CD8 subsets and VL reduction was found. That is, higher levels of Ki-67 pre-treatment corresponded with poorer patient response. The correlations were characterized by r²=0.272 for the CD3+ subset (p=0.004)(FIG. 2A), r²=0.19 for CD3+CD4+ (p=0.018)(FIG. 2B) and r²=0.21 for CD3+CD8+ (p=0.013)(FIG. 2C) subsets, respectively. The number of Ki-67+ T-cell correlated with VL reduction better than the currently accepted CD4 counts and CD38 expression levels. That is, CD4 count and CD38 expression levels pre-treatment did not predict patient response as well as Ki-67 expression level.

### Example 2

### Cohort 2, RIGHT702 Study

Methods: Blood samples from 7 patients from RIGHT 702 study were analyzed by multicolor flow cytometry for activation and proliferation T cell markers. Pearson correlation analysis was performed for baseline values of KI67 versus HIV viral load (VL) and VL changes.

Results: A negative correlation between baseline expression levels of Ki-67 in either CD3, CD4 or CD8 subsets and VL reduction was found both for short-term and long-term therapy outcomes. That is, higher levels of Ki-67 expression before treatment had begun corresponded with poorer patient response for up to 48 weeks.

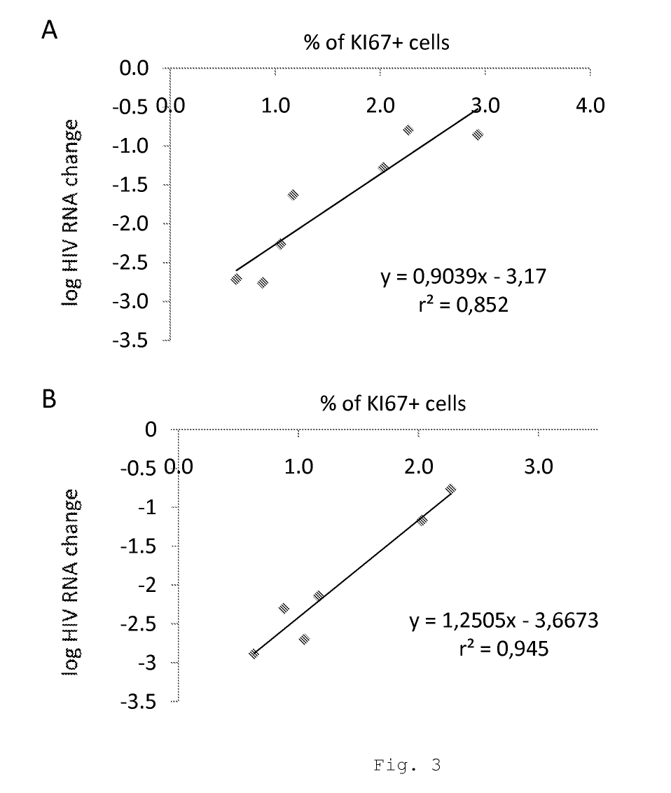

For the CD3+ subset the correlations were characterized by r²=0.852 for short-term (p=0.004)(FIG. 3A) and by r²=0.945 for the long-term therapy outcome (p=0.001)(FIG. 3B), respectively. Same negative correlation were found for the baseline Ki-67+ with VL in CD4 and CD8 subsets.

What is claimed is:

1. A diagnostic assay for Ki-67 monitoring in HIV-1 infected subjects, the steps comprising:
a. Isolation of PBMS from donor blood
b. Staining T-cells for surface markers
c. Intracellular staining T cells for Ki-67
d. Measuring Ki-67 level by flow cytometry.

2. The assay of claim 1, wherein the cells are freshly derived or frozen

3. The assay of claim 1, wherein CD3 cells were sorted from blood

4. The assay of claim 3, wherein the Ki-67 staining is performed in cell lysates.

5. The assay of claim 4, wherein the Ki-67 detection is performed by any other method, i.e. confocal microscopy, imaging, ELISA or multiplexed bead array (CBA, Luminex)

6. A diagnostic assay for immune system activation in humans, comprising quantification of expression of Ki-67 glycoprotein in one or more of the group consisting of CD3, CD4 and CD8 subsets of human PBMC.

7. The assay of claim 6, wherein the subset is the total CD3 set of human PBMC.

8. The assay of claim 6, wherein the expression of Ki-67 glycoprotein is quantified for cells in the S phase of cellular proliferation.
